(19) United States
(12) Patent Application Publication
(10) Pub. No.: US 2024/0252416 A1
(43) Pub. Date: Aug. 1, 2024

(54) COSMETIC COMPOSITION COMPRISING AT LEAST ONE ANIONIC SURFACTANT, ONE AMPHOTERIC OR ZWITTERIONIC SURFACTANT, ONE ASSOCIATIVE POLYMER AND ONE POLYOL

(71) Applicant: L'OREAL, Paris (FR)

(72) Inventors: Charlotte ROULET, SAINT-OUEN (FR); Koudedji SOW-KEBE, SAINT-OUEN (FR); Vincent CLAIREAUX, SAINT-OUEN (FR); Laurent CHESNEAU, SAINT-OUEN (FR)

(21) Appl. No.: 18/290,064

(22) PCT Filed: May 12, 2022
(86) PCT No.: PCT/EP2022/062974

§ 371 Date: Nov. 9, 2023

(30) Foreign Application Priority Data
May 12, 2021 (FR) FR2105048

(51) Int. Cl. A61K 8/73; A61K 8/34; A61K 8/39; A61K 8/44; A61Q 5/12

(52) U.S. Cl. CPC A61K 8/731; A61K 8/345; A61K 8/39; A61K 8/442; A61Q 5/12; A61K 2800/5424; A61K 2800/5428; A61K 2800/548

(57) ABSTRACT

The present invention relates to a cosmetic composition comprising: a) at least one anionic surfactant chosen from polyoxy alkylenated alkyl(amido)ether carboxy lic acids and salts thereof: b) at least one amphoteric or zwitterionic surfactant: c) at least one associative polymer: and d) at least 2% by weight, relative to the total weight of the composition, of one or more polyols.

The present invention relates to a cosmetic composition comprising at least one particular anionic surfactant, at least one amphoteric or zwitterionic surfactant, at least one associative polymer and at least one polyol in a particular content.

The invention also relates to the use of said composition for the cosmetic treatment of keratin fibres and to a cosmetic treatment process using this composition.

### TECHNICAL FIELD

Hair is generally damaged and embrittled by the action of external atmospheric agents such as light and bad weather, and also by mechanical or chemical treatments, such as brushing, combing, dyeing, bleaching, permanent-waving and/or relaxing, or even repeated washing.

Hair is thus damaged by these various factors and may over time become dry, coarse, brittle or dull, notably in fragile areas, and more particularly at the ends.

Thus, to overcome these drawbacks, it is common practice to resort to haircare treatments using compositions that condition the hair, giving it satisfactory cosmetic properties, notably in terms of smoothness, sheen, softness, suppleness, lightness, a natural feel and good disentangling properties.

These haircare compositions intended to be applied regularly to the hair may be, for example, hair conditioners, masks or sera, and may be in the form of gels, hair lotions or care creams that are more or less thick.

The products present on the market generally have a thick texture and are difficult to apply and to spread over the entire head of hair. The user thus finds these products rather unpleasant to use.

Now, users are in search of haircare products that are easy to apply and pleasant to use, while at the same time affording good sensory and care properties to the keratin fibres.

It has been discovered, surprisingly, that a cosmetic composition comprising at least one anionic surfactant chosen from polyoxyalkylenated alkyl(amido)ether carboxylic acids and salts thereof, at least one amphoteric or zwitterionic surfactant, at least one associative polymer and at least one polyol in a particular content makes it possible to achieve the objectives presented above.

Specifically, the composition according to the invention affords care that is advantageously transparent, and has good working qualities, such as a pleasant texture. The composition is also easy to spread over all the keratin fibres and is highly rinseable.

### DISCLOSURE OF THE INVENTION

A subject of the present invention is thus a cosmetic composition comprising:
a) at least one anionic surfactant chosen from polyoxyalkylenated alkyl(amido)ether carboxylic acids and salts thereof;
b) at least one amphoteric or zwitterionic surfactant;
c) at least one associative polymer; and
d) at least 2% by weight, relative to the total weight of the composition, of one or more polyols.

The composition according to the invention makes it possible to provide good cosmetic properties to keratin fibres, such as a homogeneous smooth and soft feel, sheen and suppleness without softening of the keratin fibres, such as the hair, so as to give the hair vitality, tonicity, volume, cleanliness and a healthy visual appearance. The keratin fibres are also easy to disentangle and are not made heavy by the composition.

A subject of the present invention is also a cosmetic treatment process, preferably a haircare process, notably for washing and/or conditioning keratin fibres, in particular human keratin fibres such as the hair, comprising the application to said keratin fibres of the composition according to the invention.

The present invention also relates to the use of a composition according to the invention for cosmetic treatment, notably for washing and/or conditioning keratin fibres, in particular human keratin fibres such as the hair.

Other subjects, characteristics, aspects and advantages of the invention will emerge even more clearly on reading the description and the examples that follow.

In the text hereinbelow, unless otherwise indicated, the limits of a range of values are included in that range, notably in the expressions “between” and “ranging from . . . to . . . ”.

Moreover, the expression “at least one” used in the present description is equivalent to the expression “one or more”.

### Anionic Surfactants

As indicated previously, the composition according to the invention comprises a) one or more anionic surfactants chosen from polyoxyalkylenated alkyl(amido)ether carboxylic acids and salts thereof.

The term “anionic surfactant” means a surfactant including, as ionic or ionizable groups, only anionic groups.

In the present description, a species is termed as being “anionic” when it bears at least one permanent negative charge or when it can be ionized as a negatively charged species, under the conditions of use of the composition of the invention (for example the medium or the pH) and not comprising any cationic charge.

Preferably, the anionic surfactant(s) are chosen from polyoxyalkylenated alkyl(amido) ether carboxylic acids and salts thereof, including from 2 to 50 alkylene oxide and in particular ethylene oxide groups, such as the compounds sold by the company Kao under the Akypo names.

The polyoxyalkylenated alkyl(amido)ether carboxylic acids that may be used are preferably chosen from those of formula (1):

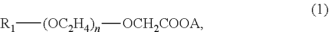

R₁ represents a linear or branched C₆-C₂₄ alkyl or alkenyl radical, a (C₈-C₉)alkylphenyl radical, a radical R₂CONH—CH₂—CH₂— with R₂ denoting a linear or branched C₉-C₂₁ alkyl or alkenyl radical;
preferably, R₁ is a C₈-C₂₀ and preferably C₈-C₁₈ alkyl radical, and aryl preferably denotes phenyl,
n is an integer or decimal number (mean value) ranging from 2 to 24 and preferably from 2 to 10,
A denotes H, ammonium, Na, K, Li, Mg or a monoethanolamine or triethanolamine residue.

Use may also be made of mixtures of compounds of formula (1), in particular mixtures of compounds bearing different groups R₁.

The polyoxyalkylenated alkyl(amido) ether carboxylic acids that are particularly preferred are those of formula (1) in which:
R₁ denotes a C₁₂-C₁₄ alkyl, lauryl, cocoyl, oleyl, nonylphenyl or octylphenyl radical;
A denotes a hydrogen or sodium atom, and
n ranges from 2 to 20, preferably from 2 to 10.

Even more preferentially, use is made of the compounds of formula (1) in which R₁ denotes a C₁₂ alkyl radical, A denotes a hydrogen or sodium atom and n ranges from 2 to 10.

Use is preferably made of polyoxyalkylenated (C₆-C₂₄)alkyl ether carboxylic acids and salts thereof, polyoxyalkylenated (C₆-C₂₄)alkylamido ether carboxylic acids, in particular those including from 2 to 15 alkylene oxide groups, salts thereof, and mixtures thereof.

When the anionic surfactant is in salt form, said salt may be chosen from alkali metal salts, such as the sodium or potassium salt, ammonium salts, amine salts and in particular amino alcohol salts, and alkaline-earth metal salts, such as the magnesium salt.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular sodium or magnesium salts are preferably used.

Among the commercial products that may preferably be used are the products sold by the company KAO under the names:
Akypo® NP 70 (R₁=nonylphenyl, n=7, A=H),
Akypo® NP 40 (R₁=nonylphenyl, n=4, A=H),
Akypo® OP 40 (R₁=octylphenyl, n=4, A=H),
Akypo® OP 80 (R₁=octylphenyl, n=8, A=H),
Akypo® OP 190 (R₁=octylphenyl, n=19, A=H),
Akypo® RLM 38 (R₁=(C₁₂-C₁₄)alkyl, n=4, A=H),
Akypo® RLM 38 NV (R₁=(C₁₂-C₁₄)alkyl, n=4, A=Na),
Akypo® RLM 45 CA (R₁=(C₁₂-C₁₄)alkyl, n=4.5, A=H)
Akypo® RLM 45 NV (R₁=(C₁₂-C₁₄)alkyl, n=4.5, A=Na),
Akypo® RLM 100 (R₁=(C₁₂-C₁₄)alkyl, n=10, A=H),
Akypo® RLM 100 NV (R₁=(C₁₂-C₁₄)alkyl, n=10, A=Na),
Akypo® RLM 130 (R₁=(C₁₂-C₁₄)alkyl, n=13, A=H),
Akypo® RLM 160 NV (R₁=(C₁₂-C₁₄)alkyl, n=16, A=Na),
or by the company Sandoz under the names:
Sandopan DTC-Acid (R₁=(C₁₃)alkyl, n=6, A=H),
Sandopan DTC (R₁=(C₁₃)alkyl, n=6, A=Na),
Sandopan LS 24 (R₁=(C₁₂-C₁₄)alkyl, n=12, A=Na),
Sandopan JA 36 (R₁=(C₁₃)alkyl, n=18, A=H),
   and more particularly the products sold under the following names:
AKYPO® RLM 45 (INCI: Laureth-5 carboxylic acid),
AKYPO® RLM 100,
AKYPO® RLM 38.

Polyoxyalkylenated (C₆-C₂₄)alkyl ether carboxylic acids and salts thereof are preferably used.

Advantageously, the total content of the anionic surfactant(s) chosen from polyoxyalkylenated (C₆-C₂₄)alkyl ether carboxylic acids and salts thereof ranges from 0.1% to 30% by weight, preferably from 0.5% to 25% by weight, more preferentially from 1% to 20% by weight, better still from 2% to 10% by weight and even better still from 3% to 8% by weight, relative to the total weight of the composition.

The composition according to the invention may optionally comprise one or more anionic surfactants other than the anionic surfactants a) as defined above.

Preferably, the composition according to the invention is free of anionic surfactants other than the anionic surfactants a) as defined above.

Preferably, the total content of anionic agent(s) a) chosen from polyoxyalkylenated alkyl(amido) ether carboxylic acids and salts thereof ranges from 0.1% to 30% by weight, preferably from 0.5% to 25% by weight, more preferentially from 1% to 20% by weight, better still from 2% to 10% by weight, even better still from 3% to 8% by weight relative to the total weight of the composition.

### Amphoteric or Zwitterionic Surfactants

The composition according to the invention also comprises b) at least one amphoteric or zwitterionic surfactant.

In particular, the amphoteric or zwitterionic surfactant(s), which are preferably non-silicone, used in the composition according to the present invention may notably be derivatives of optionally quaternized aliphatic secondary or tertiary amines, in which derivatives the aliphatic group is a linear or branched chain including from 8 to 22 carbon atoms, said amine derivatives containing at least one anionic group, for instance a carboxylate, sulfonate, sulfate, phosphate or phosphonate group.

Mention may in particular be made of (C₈-C₂₀)alkylbetaines, (C₈-C₂₀)alkylsulfobetaines, (C₈-C₂₀)alkylamido(C₃-C₈)alkylbetaines and (C₈-C₂₀)alkylamido(C₆-C₅)alkylsulfobetaines, and mixtures thereof.

Among the optionally quaternized derivatives of secondary or tertiary aliphatic amines that can be used, as defined above, mention may also be made of the compounds having the respective structures (I) and (II) below:

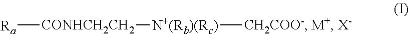

in which formula (I):
Rₐ represents a C₁₀ to C₃₀ alkyl or alkenyl group derived from an acid RaCOOH preferably present in hydrolysed coconut kernel oil; preferably, Rₐ represents a heptyl, nonyl or undecyl group;
R_{b} represents a β-hydroxyethyl group;
R_{c} represents a carboxymethyl group;
M⁺ represents a cationic counterion derived from an alkali metal or alkaline-earth metal, such as sodium, an ammonium ion or an ion derived from an organic amine; and
X″ represents an organic or mineral anionic counterion, such as that chosen from halides, acetates, phosphates, nitrates, (C₁-C₄)alkyl sulfates, (C₁-C₄)alkyl- or (C₁-C₄)alkylaryl-sulfonates, in particular methyl sulfate and ethyl sulfate; or alternatively M⁺ and X are absent;

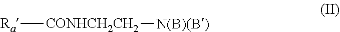

in which formula (II):
B represents the group —CH₂CH₂OX′;
B′ represents the group —(CH₂)_{z}Y′, with z=1 or 2;
X′ represents the group —CH₂COOH, —CH₂—COOZ′, —CH₂CH₂COOH or CH₂CH₂—COOZ′, or a hydrogen atom;
Y′ represents the group —COOH, —COOZ′ or —CH₂CH(OH)SO₃H or the group CH₂CH(OH)SO₃—Z′;
Z′ represents a cationic counterion derived from an alkali metal or alkaline-earth metal, such as sodium, an ammonium ion or an ion derived from an organic amine;
Rₐ represents a C₁₀ to C₃₀ alkyl or alkenyl group of an acid Rₐ—COOH which is preferably present in coconut kernel oil or in hydrolysed linseed oil, preferably Rₐ′ an alkyl group, notably a C₁₇ group, and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C₂M CONC NP Concentrate. Use may also be made of compounds of formula (III):

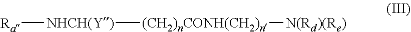

in which formula (III):
Y″ represents the group —COOH, —COOZ″ or —CH₂—CH(OH)SO₃H or the group CH₂CH(OH)SO₃—Z″;
Rₐ and Re, independently of each other, represent a C₁ to C₄ alkyl or hydroxyalkyl radical;
Z″ represents a cationic counterion derived from an alkali metal or alkaline-earth metal, such as sodium, an ammonium ion or an ion derived from an organic amine;
Rₐ″ represents a C₁₀ to C₃₀ alkyl or alkenyl group of an acid Rₐ″—COOH which is preferably present in coconut kernel oil or in hydrolysed linseed oil; and
n and n′ denote, independently of each other, an integer ranging from 1 to 3.

Among the compounds of formula (I), mention may be made of the compound classified in the CTFA dictionary under the name sodium diethylaminopropyl cocoaspartamide and sold by the company Chimex under the name Chimexane HB.

These compounds may be used alone or as a mixture.

Among the amphoteric or zwitterionic surfactants mentioned above, use is advantageously made of (C₈-C₂₀)alkylbetaines, such as cocoyl betaine, (C₈-C₂₀)alkylamido(C₃-C₈)alkylbetaines, such as cocamidopropylbetaine, (C₈-C₂₀)alkylamphoacetates, (C₈-C₂₀)alkylamphodiacetates and mixtures thereof; and preferably (C₈-C₂₀)alkylbetaines, (C₈-C₂₀)alkylamido(C₃-C₈)alkylbetaines and mixtures thereof.

Preferentially, the amphoteric or zwitterionic surfactant(s) are chosen from (C₈-C₂₀)alkylbetaines, (C₈-C₂₀)alkylamido(C₃-C₈)alkylbetaines and mixtures thereof, better still from (C₈-C₂₀)alkylamido(C₃-C₈)alkylbetaines and mixtures thereof.

Advantageously, the total content of the amphoteric or zwitterionic surfactant(s) ranges from 0.1% to 30% by weight, preferably from 0.5% to 20% by weight, more preferentially from 0.75% to 10% by weight, and better still from 1% to 6% by weight, relative to the total weight of the composition.

The total content of anionic surfactants a) and of amphoteric or zwitterionic surfactants b), present in the composition according to the invention, is preferably less than or equal to 15% by weight, and more preferentially less than or equal to 10% by weight, relative to the total weight of the composition.

### Associative Polymers

As indicated previously, the cosmetic composition according to the invention comprises c) at least one associative polymer.

It is recalled that “associative polymers” are polymers that are capable, in an aqueous medium, of reversibly associating with each other or with other molecules.

Their chemical structure more particularly comprises at least one hydrophilic zone and at least one hydrophobic zone.

The term “hydrophobic group” means a radical or polymer with a saturated or unsaturated, linear or branched hydrocarbon-based chain, comprising at least 8 carbon atoms, preferably from 8 to 30 carbon atoms, in particular from 12 to 30 carbon atoms and more preferentially from 18 to 30 carbon atoms.

Preferentially, the hydrocarbon-based group is derived from a monofunctional compound. By way of example, the hydrophobic group may be derived from a fatty alcohol such as stearyl alcohol, dodecyl alcohol or decyl alcohol. It may also denote a hydrocarbon-based polymer, for instance polybutadiene.

Preferably, the associative polymer(s) are cationic.

Among the associative cationic polymers that may be used, mention may be made, alone or as a mixture, of:
(A) cationic associative polyurethanes, which may be represented by the general formula (Ia) below:

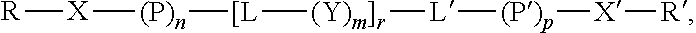

R and R′, which may be identical or different, represent a hydrophobic group or a hydrogen atom;
X and X′, which may be identical or different, represent a group including an amine function optionally bearing a hydrophobic group, or alternatively a group L″;
L, L′ and L″, which may be identical or different, represent a group derived from a diisocyanate;
P and P′, which may be identical or different, represent a group including an amine function optionally bearing a hydrophobic group;
Y represents a hydrophilic group;
r is an integer ranging from 1 to 100, preferably from 1 to 50, in particular from 1 to 25;
n, m and p each, independently of each other, range from 0 to 1000;
the molecule containing at least one protonated or quaternized amine function and at least one hydrophobic group.

Preferably, the only hydrophobic groups are the groups R and R′ at the chain ends.

One preferred family of cationic associative polyurethanes is the one corresponding to formula (Ia) described above, in which:
R and R′ both independently represent a hydrophobic group,
X and X′ each represent a group L″,
n and p are integers that are between 1 and 1000 inclusive, and L, L′, L″, P, P′, Y and
m have the meaning indicated above.

Another preferred family of cationic associative polyurethanes is the one corresponding to formula (Ia) above in which:
n=p=0 (the polymers do not include any units derived from a monomer containing an amine function, incorporated into the polymer during the polycondensation),
the protonated amine functions result from the hydrolysis of excess isocyanate functions, at the chain end, followed by alkylation of the primary amine functions formed with alkylating agents containing a hydrophobic group, i.e. compounds of the type RQ or R′Q, in which R and R′ are as defined above and Q denotes a leaving group such as a halide or a sulfate.

Yet another preferred family of cationic associative polyurethanes is the one corresponding to formula (Ia) above in which:
R and R′ both independently represent a hydrophobic group,
X and X′ both independently represent a group including a quaternary amine,
n=p=0, and
L, L′, Y and m have the meaning given above.

The number-average molecular mass (Mn) of the cationic associative polyurethanes is preferably between 400 and 500000 inclusive, in particular between 1000 and 400000 inclusive and ideally between 1000 and 300000 inclusive.

The term “hydrophobic group” means a radical or polymer containing a saturated or unsaturated, linear or branched hydrocarbon-based chain, which may contain one or more heteroatoms such as P, O, N or S, or a radical containing a perfluoro or silicone chain. When the hydrophobic group denotes a hydrocarbon-based radical, it includes at least 10 carbon atoms, preferably from 10 to 30 carbon atoms, in particular from 12 to 30 carbon atoms and more preferentially from 18 to 30 carbon atoms.

Preferentially, the hydrocarbon-based group is derived from a monofunctional compound.

By way of example, the hydrophobic group may be derived from a fatty alcohol such as stearyl alcohol, dodecyl alcohol or decyl alcohol. It may also denote a hydrocarbon-based polymer, for instance polybutadiene.

When X and/or X′ denote(s) a group including a tertiary or quaternary amine, X and/or X′ may represent one of the following formulae:

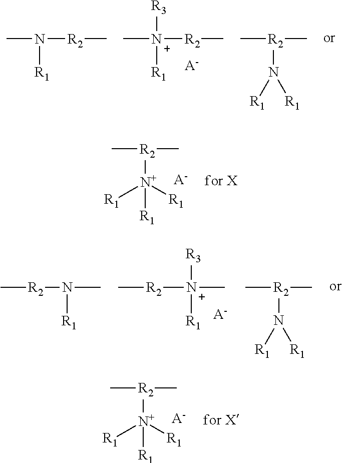

in which:
R₂ represents a linear or branched alkylene radical containing from 1 to 20 carbon atoms, optionally including a saturated or unsaturated ring, or an arylene radical, one or more of the carbon atoms possibly being replaced with a heteroatom chosen from N, S, O and P;
R₁ and R₃, which may be identical or different, represent a linear or branched C₁-C₃₀ alkyl or alkenyl radical or an aryl radical, at least one of the carbon atoms possibly being replaced with a heteroatom chosen from N, S, O and P;
A— is a physiologically acceptable anionic counterion such as a halide, for instance chloride or bromide, or mesylate.

The groups L, L′ and L″ represent a group of formula:

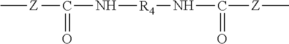

in which:
Z represents —O—, —S— or —NH—; and
R₄ represents a linear or branched alkylene radical containing from 1 to 20 carbon atoms, optionally including a saturated or unsaturated ring, or an arylene radical, one or more of the carbon atoms possibly being replaced with a heteroatom chosen from N, S, O and P.

The groups P and P′ comprising an amine function may represent at least one of the following formulae:

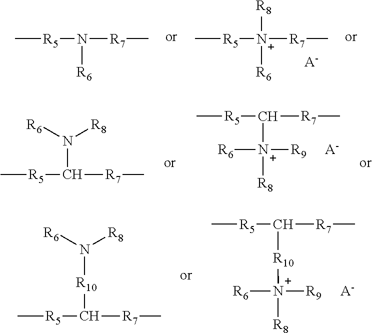

in which:
R₅ and R₇ have the same meanings as R₂ defined previously;
R₆, R₈ and Ry have the same meanings as R₁ and R₃ defined previously;
R₁₀ represents a linear or branched, optionally unsaturated alkylene group possibly containing one or more heteroatoms chosen from N, O, S and P; and
A— is a physiologically acceptable anionic counterion such as a halide, for instance chloride or bromide, or mesylate.

As regards the meaning of Y, the term “hydrophilic group” means a polymeric or non-polymeric water-soluble group.

By way of example, when it is not a polymer, mention may be made of ethylene glycol, diethylene glycol and propylene glycol.

When it is a hydrophilic polymer, in accordance with one preferred embodiment, mention may be made, for example, of polyethers, sulfonated polyesters, sulfonated polyamides or a mixture of these polymers. The hydrophilic compound is preferentially a polyether and notably a poly(ethylene oxide) or poly(propylene oxide).

The cationic associative polyurethanes of formula (Ia) according to the invention are formed from diisocyanates and from various compounds bearing functions containing labile hydrogen. The functions containing labile hydrogen may be alcohol, primary or secondary amine or thiol functions, giving, after reaction with the diisocyanate functions, polyurethanes, polyureas and polythioureas, respectively. In the present invention, the term “polyurethanes” encompasses these three types of polymer, namely polyurethanes per se, polyureas and polythioureas, and also copolymers thereof.

A first type of compound involved in the preparation of the polyurethane of formula (Ia) is a compound including at least one unit bearing an amine function. This compound may be multifunctional, but the compound is preferentially difunctional, that is to say that, according to a preferential embodiment, this compound includes two labile hydrogen atoms borne, for example, by a hydroxyl, primary amine, secondary amine or thiol function. A mixture of multifunctional and difunctional compounds in which the percentage of multifunctional compounds is low may also be used.

As mentioned above, this compound may include more than one unit containing an amine function. In this case, it is a polymer bearing a repetition of the unit containing an amine function.

Compounds of this type may be represented by one of the following formulae:

HZ—(P)ₙ—ZH or HZ—(P′)ₚ—ZH, in which Z, P. P′, n and p are as defined above.

Examples that may be mentioned include N-methyldiethanolamine, N-tert-butyldiethanolamine and N-sulfoethyldiethanolamine.

The second compound included in the preparation of the polyurethane of formula (Ia) is a diisocyanate corresponding to the formula:

O═C═N—R₄—N═C═O in which R₄ is as defined above.

By way of example, mention may be made of methylenediphenyl diisocyanate, methylenecyclohexane diisocyanate, isophorone diisocyanate, tolylene diisocyanate, naphthalene diisocyanate, butane diisocyanate and hexane diisocyanate.

A third compound involved in the preparation of the polyurethane of formula (Ia) is a hydrophobic compound intended to form the terminal hydrophobic groups of the polymer of formula (Ia).

This compound is formed form a hydrophobic group and a function containing a labile hydrogen, for example a hydroxyl, primary or secondary amine, or thiol function.

By way of example, this compound may be a fatty alcohol such as stearyl alcohol, dodecyl alcohol or decyl alcohol. When this compound includes a polymeric chain, it may be, for example, α-hydroxylated hydrogenated polybutadiene.

The hydrophobic group of the polyurethane of formula (Ia) may also result from the quaternization reaction of the tertiary amine of the compound including at least one tertiary amine unit. Thus, the hydrophobic group is introduced via the quaternizing agent. This quaternizing agent is a compound of the type RQ or R′Q, in which R and R′ are as defined above and Q denotes a leaving group such as a halide, a sulfate, etc.

The cationic associative polyurethane may also comprise a hydrophilic block. This block is provided by a fourth type of compound involved in the preparation of the polymer. This compound may be multifunctional. It is preferably difunctional. It is also possible to have a mixture in which the percentage of multifunctional compound is low.

The functions containing labile hydrogen are alcohol, primary or secondary amine or thiol functions. This compound may be a polymer terminated at the chain ends with one of these functions containing labile hydrogen.

By way of example, when it is not a polymer, mention may be made of ethylene glycol, diethylene glycol and propylene glycol.

When it is a hydrophilic polymer, mention may be made, for example, of polyethers, sulfonated polyesters and sulfonated polyamides, or a mixture of these polymers. The hydrophilic compound is preferentially a polyether and notably a poly(ethylene oxide) or poly(propylene oxide).

The hydrophilic group termed Y in formula (Ia) is optional. Specifically, the units containing a quaternary or protonated amine function may suffice to provide the solubility or water-dispersibility required for this type of polymer in an aqueous solution.

Although the presence of a hydrophilic group Y is optional, cationic associative polyurethanes including such a group are, however, preferred.
(B) quaternized cellulose derivatives, in particular quaternized celluloses modified with groups including at least one fatty chain, such as linear or branched alkyl groups, linear or branched arylalkyl groups, or linear or branched alkylaryl groups, preferably linear or branched alkyl groups, these groups including at least 8 carbon atoms, notably from 8 to 30 carbon atoms, better still from 10 to 24, or even from 10 to 14, carbon atoms; or mixtures thereof.

Preferably, mention may be made of quaternized hydroxyethylcelluloses modified with groups including at least one fatty chain, such as linear or branched alkyl groups, linear or branched arylalkyl groups, or linear or branched alkylaryl groups, preferably linear or branched alkyl groups, these groups including at least 8 carbon atoms, notably from 8 to 30 carbon atoms, better still from 10 to 24 or even from 10 to 14 carbon atoms; or mixtures thereof.

Preferentially, mention may be made of the hydroxyethylcelluloses of formula (Ib):

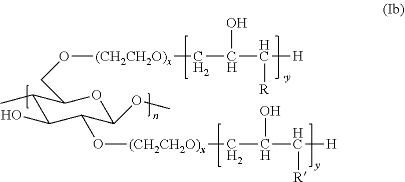

in which:
R represents an ammonium group RₐR_{b}R_{c}N⁺—, Q in which Rₐ, R_{b} and R_{c}, which may be identical or different, represent a hydrogen atom or a linear or branched C₁-C₃₀ alkyl, and Q represents an anionic counterion such as a halide, for instance a chloride or bromide; preferably an alkyl;
R′ represents an ammonium group R′ₐR′_{b}R′_{c}N⁺—, Q′ in which R′ a, R′b and R′c, which may be identical or different, represent a hydrogen atom or a linear or branched C₁-C₃₀ alkyl, and Q′ represents an anionic counterion such as a halide, for instance a chloride or bromide; preferably an alkyl;
it being understood that at least one of the radicals Rₐ, R_{b}, R_{c}, R′ a, R′b and R′c represents a linear or branched C₈-C₃₀ alkyl;
n, x and y, which may be identical or different, represent an integer of between 1 and 10000.

Preferably, in formula (Ib), at least one of the radicals Rₐ, R_{b}, R_{c}, R′ a, R′b, or R′c represents a linear or branched C₈-C₃₀, better still C₁₀-C₂₄ or even C₁₀-C₁₄ alkyl; mention may be made in particular of the dodecyl radical (C₁₂). Preferably, the other radical(s) represent a linear or branched C₁-C₄ alkyl, notably methyl.

Preferably, in formula (Ib), only one of the radicals Rₐ, R_{b}, R_{c}, R′ a, R′b or R′c represents a linear or branched C₈-C₃₀, better still C₁₀-C₂₄ or even C₁₀-C₁₄ alkyl; mention may be made in particular of the dodecyl radical (C₁₂). Preferably, the other radicals represent a linear or branched C₁-C₄ alkyl, notably methyl.

Even better still, R may be a group chosen from —N⁺(CH₃)₃, Q′ and —N⁺(C₁₂H₂₅)(CH₃)₂, Q′, preferably a group —N⁺(CH₃)₃, Q′.

Even better still, R′ may be a group —N⁺(C₁₂H₂₅)(CH₃)₂, Q″. The aryl radicals preferably denote phenyl, benzyl, naphthyl or anthryl groups.

Mention may notably be made of the polymers having the following INCI names:
Polyquaternium-24, such as the product Quatrisoft LM 200®, sold by the company Amerchol/Dow Chemical;
PG-Hydroxyethylcellulose Cocodimonium Chloride, such as the product Crodacel QM®;
PG-Hydroxyethylcellulose Lauryldimonium Chloride (C₁₂ alkyl), such as the product Crodacel QL®; and
PG-Hydroxyethylcellulose Stearyldimonium Chloride (C₁₈ alkyl), such as the product Crodacel QS®, sold by the company Croda.

Mention may also be made of the hydroxyethylcelluloses of formula (Ib) in which R represents a trimethylammonium halide and R′ represents a dimethyldodecylammonium halide; preferentially R represents trimethylammonium chloride (CH₃)₃N⁺—, C₁— and R′ represents dimethyldodecylammonium chloride (CH₃)₂(C₁₂H₂₅)N⁺—, C₁. This type of polymer is known under the INCI name Polyquaternium-67; as commercial products, mention may be made of the Softcat Polymer SL® polymers, such as SL-100, SL-60, SL-30 and SL-5, from the company Amerchol/Dow Chemical.

More particularly, the polymers of formula (Ib) are, for example, those whose viscosity is between 2000 and 3000 cPs inclusive, preferentially between 2700 and 2800 cPs. Typically, Softcat Polymer SL-5 has a viscosity of 2500 cPs, Softcat Polymer SL-30 has a viscosity of 2700 cPs, Softcat Polymer SL-60 has a viscosity of 2700 cPs and Softcat Polymer SL-100 has a viscosity of 2800 cPs. Use may also be made of Softcat Polymer SX-1300X with a viscosity of between 1000 and 2000 cPs.
(C) cationic polyvinyllactams, notably those comprising:
a) at least one monomer of vinyllactam or alkylvinyllactam type;
b) at least one monomer of structure (Ic) or (IIc) below:

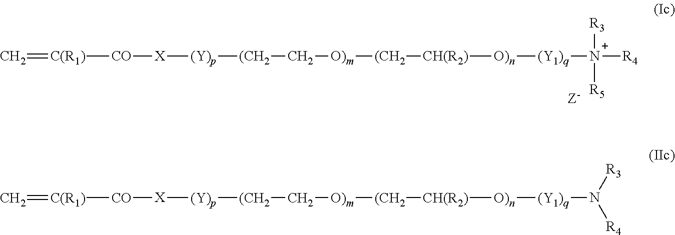

in which:
X denotes an oxygen atom or a radical NR₆,
R₁ and R₆ denote, independently of each other, a hydrogen atom or a linear or branched C₁-C₅ alkyl radical,
R₂ denotes a linear or branched C₁-C₄ alkyl radical,
R₃, R₄ and R₅ denote, independently of each other, a hydrogen atom, a linear or branched C₁-C₃₀ alkyl radical or a radical of formula (IIIc):

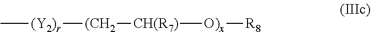

Y, Y₁ and Y₂ denote, independently of each other, a linear or branched C₂-C₁₆ alkylene radical,
R₇ denotes a hydrogen atom or a linear or branched C₁-C₄ alkyl radical or a linear or branched C₁-C₄ hydroxyalkyl radical,
R₈ denotes a hydrogen atom or a linear or branched C₁-C₃₀ alkyl radical,
p, q and r denote, independently of each other, 0 or 1,
m and n denote, independently of each other, an integer ranging from 0 to 100 inclusive,
x denotes an integer ranging from 1 to 100 inclusive,
Z denotes an anionic counterion of an organic or mineral acid, such as a halide, for instance chloride or bromide, or mesylate;
with the proviso that:
at least one of the substituents R₃, R₄, R₅ or R₈ denotes a linear or branched C₈-C₃₀ alkyl radical,
if m and/or n is other than zero, then q is equal to 1,
if m=n=0, then p or q is equal to 0.

Preferably, the counterion Z- of the monomers of formula (Ic) is chosen from halide ions, phosphate ions, the methosulfate ion and the tosylate ion.

Preferably, R₃, R₄ and R₅ denote, independently of each other, a hydrogen atom or a linear or branched C₁-C₃₀ alkyl radical.

More preferentially, the monomer b) is a monomer of formula (Ic) for which, preferentially, m and n are equal to 0.

The vinyllactam or alkylvinyllactam monomer is preferably a compound of structure (IVc):

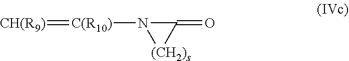

in which:
s denotes an integer ranging from 3 to 6,
R₉ denotes a hydrogen atom or a linear or branched C₁-C₅ alkyl radical,
R₁₀ denotes a hydrogen atom or a linear or branched C₁-C₅ alkyl radical, with the proviso that at least one of the radicals R₉ and R₁₀ denotes a hydrogen atom.

Even more preferentially, the monomer (IVc) is vinylpyrrolidone.

The cationic poly(vinyllactam) polymers according to the invention may also contain one or more additional monomers, preferably cationic or nonionic monomers.

As compounds that are particularly preferred, mention may be made of the following terpolymers comprising at least:
a) one monomer of formula (IVc),
b) one monomer of formula (Ic) in which p=1, m=n=q=0, R₃ and R₄ denote, independently of each other, a hydrogen atom or a C₁-C₅ alkyl radical and R₅ denotes a linear or branched C₈-C₂₄ alkyl radical, and
c) one monomer of formula (IIc) in which p=1, m=n=q=0, R₃ and R₄ denote, independently of each other, a hydrogen atom or a linear or branched C₁-C₅ alkyl radical.

Even more preferentially, terpolymers comprising, by weight, 40% to 95% of monomer (a), 0.1% to 55% of monomer (c) and 0.25% to 50% of monomer (b) will be used. Such polymers are notably described in patent application WO-00/68282.

As cationic polymer(vinyllactam) polymers according to the invention, the following are notably used:
vinylpyrrolidone/dimethylaminopropylmethacrylamide/dodecyldimethylmethacrylamidopropylammonium tosylate terpolymers,
vinylpyrrolidone/dimethylaminopropylmethacrylamide/cocoyldimethylmethacrylamidopropylammonium tosylate terpolymers,
vinylpyrrolidone/dimethylaminopropylmethacrylamide/lauryldimethylmethacrylamidopropylammonium tosylate or chloride terpolymers.

The vinylpyrrolidone/dimethylaminopropylmethacrylamide/lauryldimethylmethylacrylamidopropylammonium chloride terpolymer is notably sold by the company ISP under the names Styleze W10® and Styleze W20L® (INCI name: Polyquaternium-55).

The weight-average molecular mass (Mw) of the cationic poly(vinyllactam) polymers is preferably between 500 and 20000000, more particularly between 200000 and 2000000 and preferentially between 400000 and 800000.
(D) the cationic polymer(s) obtained by polymerization of a monomer mixture comprising one or more vinyl monomers substituted with one or more amino groups, one or more hydrophobic nonionic vinyl monomers, and one or more associative vinyl monomers, as described in patent application WO 2004/024779.

Among these copolymers, mention may be made more particularly of the products of polymerization of a monomer mixture comprising:
a di(C₁-C₄ alkyl)amino(C₁-C₆ alkyl) methacrylate,
one or more C₁-C₃₀ alkyl esters of (meth)acrylic acid,
a polyethoxylated C₁₀-C₃₀ alkyl methacrylate (20-25 mol of ethylene oxide units),
a 30/5 polyethylene glycol/polypropylene glycol allyl ether,
a hydroxy(C₂-C₆ alkyl) methacrylate, and
an ethylene glycol dimethacrylate.

Such a polymer is, for example, the compound sold by the company Lubrizol under the name Carbopol Aqua CC® and which corresponds to the INCI name Polyacrylate-1 Crosspolymer.

Advantageously, the associative polymer(s) c) are cationic and are preferentially chosen from quaternized (poly)hydroxyethylcelluloses modified with groups including at least one fatty chain, such as alkyl, arylalkyl or alkylaryl groups including at least 10 carbon atoms, or mixtures thereof.

Preferably, the associative polymer(s) c) are cationic and are preferentially chosen from quaternized (poly)hydroxyethylcelluloses modified with groups including at least one alkyl group containing at least 10 carbon atoms, preferentially ranging from 10 to 22 carbon atoms, and more preferentially ranging from 12 to 16 carbon atoms.

More preferentially, the associative polymer c) is an associative cationic polymer having the INCI name Polyquaternium-67.

Advantageously, the total content of the associative polymer(s) c) ranges from 0.01% to 10% by weight, preferably from 0.05% to 5% by weight, more preferentially from 0.1% to 3% by weight, relative to the total weight of the composition.

More advantageously, the total content of cationic associative polymer(s) c) ranges from 0.01% to 10% by weight, preferably from 0.05% to 5% by weight and more preferentially from 0.1% to 3% by weight, relative to the total weight of the composition.

### Polyols

As indicated previously, the composition according to the invention also comprises d) one or more polyols, in a total content of at least 2% by weight relative to the total weight of the composition.

For the purposes of the present invention, the term “polyol” means an organic compound constituted of a hydrocarbon-based chain optionally interrupted with one or more oxygen atoms and bearing at least two free hydroxyl groups (—OH) borne by different carbon atoms, this compound possibly being cyclic or acyclic, linear or branched, and saturated or unsaturated.

More particularly, the polyol(s) comprise from 2 to 30 hydroxyl groups, more preferentially from 2 to 10 hydroxyl groups, even more preferentially from 2 to 3 hydroxyl groups. They preferably comprise from 2 to 10 carbon atoms, notably from 2 to 8 carbon atoms and better still from 2 to 6 carbon atoms.

Advantageously, the polyol(s) are chosen from diglycerol, glycerol, propylene glycol, propane-1,3-diol, 1,3-butylene glycol, pentane-1,2-diol, octane-1,2-diol, dipropylene glycol, hexylene glycol, ethylene glycol, polyethylene glycols, sorbitol, sugars, such as glucose, and mixtures thereof, preferably glycerol.

Advantageously, the total content of polyol(s) ranges from 2% to 30% by weight, preferably from 2.5% to 20% by weight, more preferentially from 3% to 15% by weight and better still from 4% to 10% by weight relative to the total weight of the composition.

### Organic Solvents

The composition according to the invention may comprise one or more water-soluble organic solvents chosen from monoalcohols containing from 1 to 5 carbon atoms, such as ethanol, isopropanol and butanol.

The cosmetic composition according to the invention may comprise water, preferably in a content ranging from 40% to 97% by weight, better still from 60% to 95% by weight, even still better from 75% to 90% by weight relative to the total weight of the composition.

### Nonionic Surfactants

According to a preferred embodiment, the composition according to the invention may also comprise one or more nonionic surfactants.

The nonionic surfactant(s) that may be used in the composition of the present invention are described, for example, in the Handbook of Surfactants by M. R. Porter, published by Blackie & Son (Glasgow and London), 1991, pages 116-178.

Examples of nonionic surfactants that may be mentioned include the following compounds, alone or as a mixture:
oxyalkylenated (C₈-C₂₄)alkylphenols;
saturated or unsaturated, linear or branched, oxyalkylenated or glycerolated C₈ to C₄₀ alcohols, preferably including one or two fatty chains;
saturated or unsaturated, linear or branched, oxyalkylenated C₈ to C₃₀ fatty acid amides;
esters of saturated or unsaturated, linear or branched, C₈ to C₃₀ acids and of polyethylene glycols;
esters of saturated or unsaturated, linear or branched, C₈ to C₃₀ acids and of sorbitol, which are preferably oxyethylenated;
fatty acid and sucrose esters;
C₈-C₃₀ fatty acid and sorbitan esters;
oxyethylenated C₈-C₃₀ fatty acid and sorbitan esters;
(C₈-C₃₀)alkyl(poly)glucosides and (C₈-C₃₀)alkenyl(poly)glucosides, which are optionally oxyalkylenated (0 to 10 oxyalkylene units) and comprise from 1 to 15 glucose units, (C₈-C₃₀)alkyl(poly)glucoside esters;
hydrogenated or non-hydrogenated, saturated or unsaturated oxyethylenated plant oils;
condensates of ethylene oxide and/or of propylene oxide;
N—(C₈-C₃₀)alkylglucamine and N—(C₈-C₃₀)acylmethylglucamine derivatives;
amine oxides.

The nonionic surfactants may be chosen from alcohols, α-diols and (C₁-C₂₀)alkylphenols, these compounds being ethoxylated, propoxylated or glycerolated and bearing at least one fatty chain including, for example, from 8 to 24 carbon atoms and preferably from 8 to 18 carbon atoms, the number of ethylene oxide or propylene oxide groups possibly ranging notably from 1 to 200, and the number of glycerol groups possibly ranging notably from 1 to 30.

Mention may also be made of condensates of ethylene oxide and of propylene oxide with fatty alcohols; ethoxylated fatty amides preferably containing from 1 to 30 ethylene oxide units, polyglycerolated fatty amides including on average from 1 to 5, and in particular from 1.5 to 4, glycerol groups, ethoxylated fatty acid and sorbitan esters containing from 1 to 30 ethylene oxide units, fatty acid esters of sucrose, fatty acid esters of polyethylene glycol, (C₆-C₂₄ alkyl)polyglycosides, oxyethylenated plant oils, N—(C₆-C₂₄ alkyl)glucamine derivatives, amine oxides such as (C₁₀-C₁₄ alkyl)amine oxides or N—(C₁₀-C₁₄ acyl)aminopropylmorpholine oxides.

Mention may be made in particular of saturated or unsaturated, linear or branched, oxyethylenated C₈ to C₄₀ fatty alcohols comprising from 1 to 100 mol of ethylene oxide, preferably from 2 to 50, more particularly from 2 to 40 mol, or even from 3 to 20 mol of ethylene oxide and including at least one C₈-C₂₀ and notably C₁₀-C₁₈ alkyl chain; notably lauryl alcohol containing 4 mol of ethylene oxide (INCI name: Laureth-4).

The C₈-C₃₀ and preferably C₁₂-C₂₂ fatty acid and sorbitan esters (notably monoesters, diesters and triesters) may be chosen from:
sorbitan caprylate; sorbitan cocoate; sorbitan isostearate; sorbitan laurate; sorbitan oleate; sorbitan palmitate; sorbitan stearate; sorbitan diisostearate; sorbitan dioleate; sorbitan distearate; sorbitan sesquicaprylate; sorbitan sesquiisostearate; sorbitan sesquioleate; sorbitan sesquistearate; sorbitan triisostearate; sorbitan trioleate; and sorbitan tristearate.

The polyoxyethylenated C₈-C₃₀ (preferably C₁₂-C₁₈) fatty acid and sorbitan esters (notably monoesters, diesters and triesters) notably containing from 2 to 30 mol of ethylene oxide may be chosen from polyoxyethylenated esters of C₁₂-C₁₈ fatty acids, in particular lauric, myristic, cetylic or stearic acid, and of sorbitan notably containing from 2 to 30 mol and better still from 2 to 20 mol of ethylene oxide, such as:
polyoxyethylenated sorbitan monolaurate (4 OE) (POLYSORBATE-21),
polyoxyethylenated sorbitan monolaurate (20 OE) (POLYSORBATE-20),
polyoxyethylenated sorbitan monopalmitate (20 OE) (POLYSORBATE-40),
polyoxyethylenated sorbitan monostearate (20 OE) (POLYSORBATE-60),
polyoxyethylenated sorbitan monostearate (4 OE) (POLYSORBATE-61),
polyoxyethylenated sorbitan monooleate (20 OE) (POLYSORBATE-80),
polyoxyethylenated sorbitan monooleate (5 OE) (POLYSORBATE-81),
polyoxyethylenated sorbitan tristearate (20 OE) (POLYSORBATE-65),
polyoxyethylenated sorbitan trioleate (20 OE) (POLYSORBATE-85).

The hydrogenated or non-hydrogenated, saturated or unsaturated oxyethylenated plant oils may be oils such as sweet almond oil, argan oil, avocado oil, groundnut oil, camellia oil, safflower oil, beauty-leaf oil, rapeseed oil, coconut oil (or coconut kernel oil), coriander oil, marrow oil, wheat germ oil, jojoba oil, linseed oil, macadamia oil, corn germ oil, hazelnut oil, walnut oil, vernonia oil, apricot kernel oil, olive oil, evening-primrose oil, palm oil, passion flower oil, grapeseed oil, rose oil, castor oil, rye oil, sesame oil, rice bran oil, camelina oil, soybean oil, sunflower oil, pracaxi oil, babassu oil, mongongo oil, marula oil, arara oil, shea butter oil, and Brazil nut oil. In particular, hydrogenated palm oil and hydrogenated castor oil may be mentioned among the hydrogenated plant oils.

Preferably, the nonionic surfactants may be chosen from saturated or unsaturated, linear or branched, oxyethylenated C₈ to C₄₀ fatty alcohols, preferably comprising from 3 to 20 mol of ethylene oxide and including at least one C₈-C₂₀ alkyl chain, polyoxyethylenated C₈-C₃₀ fatty acid and sorbitan esters notably containing from 2 to 30 mol of ethylene oxide, hydrogenated or non-hydrogenated, saturated or unsaturated, oxyethylenated plant oils, and mixtures thereof.

Advantageously, the total content of nonionic surfactant(s), when they are present, ranges from 0.1% to 15% by weight, preferably from 0.5% to 10% by weight and more preferentially from 1% to 5% by weight relative to the total weight of the composition.

### Additives

The composition according to the invention may contain any adjuvant or additive usually used.

Among the additives that may be used, mention may be made of reducing agents, thickeners other than the associative polymers, softeners, moisturizers, UV-screening agents, peptizers, solubilizers, fragrances, proteins, vitamins, polymers other than the polymers described previously, preserving agents, oils, waxes and mixtures thereof.

A subject of the present invention is also a cosmetic treatment process, preferably a haircare process, notably for washing and/or conditioning keratin fibres, in particular human keratin fibres such as the hair, comprising the application to said keratin fibres of the composition according to the invention.

A subject of the present invention is also the use of the composition according to the invention for cosmetic treatment, notably for washing and/or conditioning keratin fibres, in particular of human keratin fibres such as the hair.

The present invention will now be described more specifically by means of examples, which do not in any way limit the scope of the invention. However, the examples make it possible to support specific characteristics, variants and preferred embodiments of the invention.

### EXAMPLES

In the examples that follow, all the amounts are given, unless otherwise indicated, as mass percentages of active material relative to the total weight of the composition.

### Example 1

Compositions A1, A2 and A3 according to the invention and comparative composition B1, as described in Table 1 below, were prepared. The amounts are expressed as grams of active material/100 g.

TABLE 1

| Compositions | A1 | B1 | A2 | A3 |
|---|---|---|---|---|
| Laureth-5 carboxylic acid | 5.7 | 5.7 | 4.1 | 4.1 |
| Cocamidopropyl betaine | 1.4 | 1.4 | 2 | 2 |
| Polyquaternium-67 | 0.5 | 0.5 | 0.45 | 0.45 |
| Sodium chloride | 0.3 | 0.3 | 0.6 | 0.6 |
| Arginine | — | — | 0.35 | — |
| Isopropyl myristate | — | — | 0.5 | 0.5 |
| PEG-40 hydrogenated castor oil | — | — | 1.5 | 1.5 |
| Laureth-4 | — | — | 0.6 | 0.6 |
| Polysorbate 20 | — | — | 0.3 | 0.3 |
| Preserving agents | qs | qs | qs | qs |
| Glycerol | 6 | 1.5 | 5 | 5 |
| Water | qs 100 | qs 100 | qs 100 | qs 100 |

The turbidity was then evaluated. The measurements were taken with a Hach 2100P model portable turbidimeter. The values are given in NTU at 25° C. The higher the value, the more opaque the sample.

The results obtained are collated in Table 2 below.

TABLE 2

| Compositions | A1 | A2 | B1 |
|---|---|---|---|
| Turbidity value at 25° C. (NTU) | 13.2 | 4.4 | 17.9 |

Compositions A1 and A2 according to the invention have low turbidity values, lower than that of the comparative composition B1.

Thus, the compositions according to the invention are more transparent than the comparative composition B1.

Moreover, compositions A1 and B1 were applied per half-head on a panel of eight people, at a rate of 6 g of composition per half-head.

The cosmetic performance qualities, notably in terms of suppleness and smoothness of feel, were evaluated at different times of use of the compositions.

It was found that:
when the composition is applied to the hair, composition A1 according to the invention gives results that are superior to those given by the comparative composition B1, in terms of suppleness (the smooth feel is not evaluated);
when the composition is rinsed off, composition A1 according to the invention gives results that are superior to those given by the comparative composition B1, in terms of suppleness and smoothness of feel;
on damp hair (before drying), composition A1 according to the invention gives results that are superior to those given by the comparative composition B1, in terms of suppleness and smoothness of feel;
on dried hair, composition A1 according to the invention gives results that are superior to those given by the comparative composition B1, in terms of suppleness and smoothness of feel; it was also found that there is better coating of the hair, better strand separation and also better visual smoothness with the invention.

1-15. (canceled)

16. A cosmetic composition comprising:
a) at least one anionic surfactant chosen from polyoxyalkylenated alkyl(amido)ether carboxylic acids, or salts thereof;
b) at least one amphoteric or zwitterionic surfactant;
c) at least one associative polymer; and
d) at least 2% by weight, relative to the total weight of the composition, of at least one polyol.

17. The composition of claim 16, wherein the polyoxyalkylenated alkyl(amido)ether carboxylic acids are chosen from compounds of formula (1): 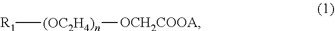
R₁ represents a linear or branched C₆-C₂₄ alkyl or alkenyl radical, a (C₈-C₉)alkylphenyl radical, a radical R₂CONH—CH₂—CH₂— wherein R₂ represents a linear or branched C₉-C₂₁ alkyl or alkenyl radical;
n is an integer or decimal number ranging from 2 to 24;
A represents H, ammonium, Na, K, Li, Mg, or a monoethanolamine or triethanolamine residue.

18. The composition of claim 16, wherein the polyoxyalkylenated alkyl(amido)ether carboxylic acids are chosen from compounds of formula (1): 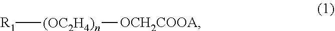
wherein:
R₁ represents a C₁₂-C₁₄ alkyl, lauryl, cocoyl, oleyl, nonylphenyl, or octylphenyl radical;
A represents a hydrogen or sodium atom; and
n represents an integer ranging from 2 to 20.

19. The composition of claim 16, wherein the total amount of anionic surfactant(s) ranges from 0.1% to 30% by weight, relative to the total weight of the composition.

20. The composition of claim 16, wherein the amphoteric or zwitterionic surfactant(s) are chosen from (C₈-C₂₀)alkylbetaines, (C₈-C₂₀)alkylsulfobetaines, (C₈-C₂₀)alkylamido(C₃-C₅)alkylbetaines, (C₈-C₂₀)alkylamido(C₆-C₅)alkylsulfobetaines, or mixtures thereof.

21. The composition of claim 16, wherein the total amount of the amphoteric or zwitterionic surfactant(s) ranges from 0.1% to 30% by weight, relative to the total weight of the composition.

22. The composition of claim 16, wherein the associative polymer(s) are cationic polymers.

23. The composition of claim 16, wherein the associative polymer(s) are cationic polymers and are chosen from quaternized (poly)hydroxyethylcelluloses modified with groups including at least one fatty chain, or mixtures thereof.

24. The composition of claim 16, wherein the total amount of associative polymer(s) ranges from 0.01% to 10% by weight relative to the total weight of the composition.

25. The composition of claim 16, wherein the polyol(s) are chosen from diglycerol, glycerol, propylene glycol, propane-1,3-diol, 1,3-butylene glycol, pentane-1,2-diol, octane-1,2-diol, dipropylene glycol, hexylene glycol, ethylene glycol, polyethylene glycols, sorbitol, sugars, or mixtures thereof.

26. The composition of claim 16, wherein the at least one polyol is glycerol.

27. The composition of claim 16, wherein the total amount of polyol(s) ranges from 2% to 30% by weight, relative to the total weight of the composition.

28. The composition of claim 16, further comprising at least one nonionic surfactant.

29. The composition of claim 28, wherein the at least one nonionic surfactant is chosen from saturated or unsaturated, linear or branched, oxyethylenated C₈ to C₄₀ fatty alcohols, or mixtures thereof.

30. The composition of claim 16, wherein the total amount of nonionic surfactant(s) ranges from 0.1% to 15% by weight, relative to the total weight of the composition.

31. A method for washing and/or conditioning keratin fibres, comprising applying to the keratin fibres at least one composition comprising:
a) at least one anionic surfactant chosen from polyoxyalkylenated alkyl(amido)ether carboxylic acids, or salts thereof;
b) at least one amphoteric or zwitterionic surfactant;
c) at least one associative polymer; and
d) at least 2% by weight, relative to the total weight of the composition, of at least one polyol.

32. The method of claim 31, wherein the polyoxyalkylenated alkyl(amido)ether carboxylic acids are chosen from compounds of formula (1): 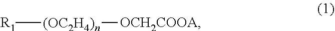
wherein:
R₁ represents a linear or branched C₆-C₂₄ alkyl or alkenyl radical, a (C₈-C₉)alkylphenyl radical, a radical R₂CONH—CH₂—CH₂— wherein R₂ represents a linear or branched C₉-C₂₁ alkyl, or alkenyl radical;
n is an integer or decimal number ranging from 2 to 24;
A represents H, ammonium, Na, K, Li, Mg, or a monoethanolamine or triethanolamine residue.

33. The method of claim 31, wherein the polyoxyalkylenated alkyl(amido)ether carboxylic acids are chosen from compounds of formula (1): 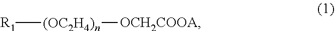
wherein:
R₁ represents a C₁₂-C₁₄ alkyl, lauryl, cocoyl, oleyl, nonylphenyl, or octylphenyl radical;
A represents a hydrogen or sodium atom; and
n represents an integer ranging from 2 to 20.

34. The method of claim 31, wherein the total amount of anionic surfactant(s) ranges from 0.1% to 30% by weight, relative to the total weight of the composition.

35. A method of applying to human keratin fibers at least one composition comprising:
a) at least one anionic surfactant chosen from polyoxyalkylenated alkyl(amido)ether carboxylic acids, or salts thereof;
b) at least one amphoteric or zwitterionic surfactant;
c) at least one associative polymer; and
d) at least 2% by weight, relative to the total weight of the composition, of at least one polyol.
